(19) United States
(12) Patent Application Publication
(10) Pub. No.: US 2021/0019886 A1
(43) Pub. Date: Jan. 21, 2021

(54) MEDICINE VERIFICATION DEVICE AND MEDICINE VERIFICATION METHOD

(71) Applicant: FUJIFILM Toyama Chemical Co., Ltd., Tokyo (JP)

(72) Inventor: Kazuchika IWAMI, Ashigarakami-gun (JP)

(73) Assignee: FUJIFILM Toyama Chemical Co., Ltd., Tokyo (JP)

(21) Appl. No.: 17/039,715

(22) Filed: Sep. 30, 2020

Related U.S. Application Data
(63) Continuation of application No. PCT/JP2019/009381, filed on Mar. 8, 2019.

(30) Foreign Application Priority Data
Apr. 3, 2018 (JP) 2018-071827

(51) Int. Cl. G06T 7/00; G16H 40/20; G16H 20/10; G16H 70/40; G06Q 30/00; G06K 9/62; A61J 1/03; A61J 1/10; G01N 21/86; G01N 21/47; G01N 21/59

(52) U.S. Cl. CPC G06T 7/0014; G16H 40/20; G16H 20/10; G16H 70/40; G06Q 30/0185; G06K 9/6201; A61J 2205/10; A61J 1/10; G01N 21/86; G01N 21/47; G01N 21/59; G06K 2209/21; A61J 1/03

(57) ABSTRACT

The type of a verification target medicine is verified using a master image and a captured image of the verification target medicine packed in a packaging material having light transmissivity, and then the characteristic information indicating an optical characteristic that influences the sharpness of the captured image is acquired for the packaging material packing the verification target medicine having the type verified to match the type of the medicine appearing in the master image. Subsequently, in a case where it is verified that the type of the verification target medicine matches the type of the medicine appearing in the master image, determination is made as to whether the optical characteristic indicated by the characteristic information satisfies a standard. When the optical characteristic satisfies the standard, the update processing for updating the registered master image to the image of the verification target medicine is executed.

### CROSS-REFERENCE TO RELATED APPLICATIONS

This application is a Continuation of PCT International Application No. PCT/JP2019/009381 filed on Mar. 8, 2019, which claims priority under 35 U.S.C. § 119(a) to Japanese Patent Application No. 2018-071827 filed on Apr. 3, 2018. The above application is hereby expressly incorporated by reference, in its entirety, into the present application.

### BACKGROUND OF THE INVENTION

The present invention relates to a medicine verification device and a medicine verification method, and more particularly relates to a medicine verification device and a medicine verification method adapted to verify the type of verification target medicine using a master image registered in association with the type of medicine and a captured image of the verification target medicine.

### RELATED ART

As an already known technique, an automatic inspection has been performed by a machine or the like as to whether a medicine is correctly packaged in a packaging bag such as a sachet sheet as instructed by a prescription. Such an automatic inspection device (hereinafter, referred to as a medicine verification device) captures an image of a medicine packed in a packaging bag, for example, within the device. Subsequently, the device verifies the type and number of the medicines appearing in the captured image.

Furthermore, for example, a verification method described in JP 2005-249615 A (in particular, refer to claim 1 and paragraphs 0021 to 0022 of JP 2005-249615 A) is used to compare a captured image of a workpiece as a verification target and an image of a non-defective workpiece being a reference image (hereinafter, a master image) to verify whether the workpiece as a verification target matches the non-defective workpiece.

Furthermore, in the verification method described in JP 2005-249615 A, the master image is updated with the captured image of the verification target workpiece determined to match the non-defective workpiece in the verification, as a new master image. With this configuration, image comparison is always performed with the latest captured image of the workpiece verified to match the non-defective workpiece, as the master image. As a result, for example, even with a difference in the illuminance of the illumination at individual timings of image capturing or with a gradual change in the illuminance, it is possible to continuously perform verification without processes of adjusting the illumination, changing the threshold, re-registering the master image, or the like. That is, according to the verification method described in JP 2005-249615 A, it is possible to perform verification more appropriately because it is less likely to be influenced by the environmental change, as compared with the case of performing fixed registration of a master image.

### SUMMARY OF THE INVENTION

In capturing an image of a verification target medicine, the medicine is imaged in a state of being packed in a packaging bag having light transmissivity (that is, through the packaging bag). Therefore, the sharpness of the captured image changes in accordance with the optical characteristics (specifically, the light transmissivity and the light scattering) of the packaging bag. On the other hand, in the case of registering the latest captured image of a workpiece verified to match a non-defective workpiece as a master image as in the verification method described in JP 2005-249615 A, for example, always registering an image of a medicine packed by a packaging bag having low light transmissivity as a master image would lead to a failure in appropriate execution of the subsequent verifications using the same master image.

The present invention has been made in view of the above circumstances and aims to provide a medicine verification device and a medicine verification method capable of appropriately updating a master image in consideration of optical characteristics of a packaging material that would influence the sharpness of an image.

In order to achieve the above object, the medicine verification device of the present invention includes: an image capturing unit configured to capture an image of a verification target medicine packed in a packaging material having light transmissivity; a verification unit configured to verify a type of the verification target medicine using a master image registered in association with the type of medicine and the image of the verification target medicine captured by the image capturing unit; a characteristic information acquisition unit configured to acquire characteristic information indicating an optical characteristic that influences sharpness of the image captured by the image capturing unit, for the packaging material that packs the verification target medicine verified by the verification unit to have a type that matches the type of the medicine appearing in the master image; and an update processing unit configured to execute update processing for updating the registered master image to the image of the verification target medicine, in which, in a case where the verification unit has verified that the type of the verification target medicine matches the type of the medicine appearing in the master image, the update processing unit determines whether the optical characteristic indicated by the characteristic information acquired by the characteristic information acquisition unit satisfies a standard for the optical characteristic, and executes the update processing when the optical characteristic satisfies the standard.

Furthermore, it is preferable to have a configuration in which the characteristic information acquisition unit acquires the characteristic information indicating light transmittance of the packaging material, and in a case where the verification unit has verified that the type of the verification target medicine matches the type of the medicine appearing in the master image, the update processing unit determines whether the light transmittance indicated by the characteristic information acquired by the characteristic information acquisition unit exceeds a threshold, and executes the update processing when the light transmittance exceeds the threshold.

Furthermore, it is preferable to have a configuration further including a measurement unit configured to measure the optical characteristic of the packaging material, in which the measurement unit is provided inside the medicine verification device, and the characteristic information acquisition unit acquires the characteristic information indicating the optical characteristic measured by the measurement unit.

Furthermore, it is also allowable to have a configuration further including a measurement unit configured to measure the optical characteristic of the packaging material, the measurement unit being provided outside the medicine verification device, in which the characteristic information acquisition unit acquires the characteristic information indicating the optical characteristic measured by the measurement unit by an input of the characteristic information indicating the optical characteristic measured by the measurement unit, to the medicine verification device.

Furthermore, it is preferable to have a configuration in which the packaging material is a bag-shaped packaging bag, further provided with a conveyance unit configured to convey a strip-shaped continuous packaging bag including continuously arranged packaging bags along a conveyance path, the image capturing unit captures an image for each of the packaging bags at an intermediate position of the conveyance path, and the measurement unit measures the optical characteristic for at least one packaging bag out of the continuous packaging bag at an intermediate position of the conveyance path.

Furthermore, it is preferable to further include a light irradiation unit configured to emit light to the packaging bag within an imaging range of the image capturing unit out of the continuous packaging bag when the image capturing unit captures the image.

Furthermore, it is preferable to have a configuration in which the continuous packaging bag includes an empty packaging bag containing no medicine, and the measurement unit measures an optical characteristic of the empty packaging bag.

Furthermore, it is preferable to have a configuration in which, in a case where the verification unit has verified that the type of the verification target medicine matches the type of the medicine appearing in the master image, the update processing unit determines whether the light transmittance indicated by the characteristic information acquired by the characteristic information acquisition unit exceeds a threshold and the light transmittance of the packaging material appearing in the master image, and executes the update processing when the light transmittance indicated by the characteristic information acquired by the characteristic information acquisition unit exceeds the threshold and the light transmittance of the packaging material appearing in the master image.

Furthermore, it is preferable to have a configuration in which, in a case where the verification unit has verified that the type of the verification target medicine matches the type of the medicine appearing in the master image, the update processing unit determines whether the light transmittance indicated by the characteristic information acquired by the characteristic information acquisition unit exceeds the threshold and whether an imaging position of the verification target medicine is closer to a center of an imaging range of the image capturing unit than the imaging position of the medicine appearing in the master image, and the update processing unit executes the update processing when the light transmittance indicated by the characteristic information acquired by the characteristic information acquisition unit exceeds the threshold and the imaging position of the verification target medicine is closer to the center of the imaging range of the image capturing unit than the imaging position of the medicine appearing in the master image.

Moreover, it is preferable that the characteristic information acquisition unit acquires characteristic information indicating at least one of the light transmissivity and the light scattering characteristic of the packaging material.

Furthermore, it is preferable that the characteristic information acquisition unit acquires the characteristic information indicating at least one of light transmittance, a haze value, and a Modulation Transfer Function curve of the packaging material, the Modulation Transfer Function curve indicating a correspondence relationship between a contrast and a spatial frequency.

Furthermore, it is preferable to have a configuration further including a prescription condition acquisition unit configured to acquire prescription condition information indicating a prescription condition set for prescribing a medicine, in which the verification unit verifies the type of the verification target medicine using the master image corresponding to the type of the medicine specified by the prescription condition information and an image of the verification target medicine captured by the image capturing unit.

In order to achieve the above object, the medicine verification method of the present invention includes: a step of capturing an image of a verification target medicine packed in a packaging material having light transmissivity by using an image capturing unit; a step of verifying a type of the verification target medicine using a master image registered in association with the type of medicine and the image of the verification target medicine captured by the image capturing unit; a step of acquiring characteristic information indicating an optical characteristic that influences sharpness of the image captured by the image capturing unit, for the packaging material that packs the verification target medicine verified to have a type that matches the type of the medicine appearing in the master image; and a step of executing update processing for updating the registered master image to the image of the verification target medicine, in which, in a case where it is verified that the type of the verification target medicine matches the type of the medicine appearing in the master image, determination is made as to whether the optical characteristic indicated by the characteristic information acquired for the packaging material packing the verification target medicine satisfies a standard for the optical characteristic, and the update processing is executed when the optical characteristic satisfies the standard.

According to the medicine verification device and the medicine verification method of the present invention, in a case where it is verified that the type of the verification target medicine matches the type of the medicine appearing in the master image, determination is made as to whether the optical characteristic of the packaging material packing the verification target medicine satisfies a standard, and the master image is updated using the captured image of the verification target medicine when the optical characteristic satisfies the standard. This makes it possible to determine the necessity of an image update in accordance with the sharpness of the captured image of the verification target medicine, and possible to update the master image in a case where the update is necessary (for example, in a case where the update of the master image will be advantageous in a subsequent verification).

### BRIEF DESCRIPTION OF THE DRAWINGS

FIG. 1 is a diagram illustrating a flow of a medicine prescription operation.

FIG. 2 is a view illustrating a continuous packaging bag.

FIG. 3 is a schematic view illustrating an internal structure of a device main body included in a medicine verification device according to one embodiment of the present invention.

FIG. 4 is a view illustrating an imaging range of an image capturing unit and a part of a continuous packaging bag included in the range.

FIG. 5 is a schematic top view of a plurality of light emitting units of a light irradiation unit.

FIG. 6 is a block diagram illustrating a configuration of a processing device included in a medicine verification device according to one embodiment of the present invention.

FIG. 7 is a diagram illustrating a general procedure of a verification flow performed by a verification unit.

FIG. 8 is a view illustrating a medicine extraction image.

FIG. 9 is a diagram illustrating a database in which master images are registered.

FIG. 10 is a diagram illustrating a master image management table.

FIG. 11 is a diagram illustrating a procedure of an update flow executed by an update processing unit.

FIG. 12 is a diagram illustrating a procedure for determining the necessity of execution of update processing.

FIG. 13 is a diagram illustrating a flow of a basic operation of a medicine verification device according to one embodiment of the present invention.

FIG. 14 is a schematic diagram illustrating a method of measuring optical characteristics of a packaging material using an integrating sphere.

FIG. 15 is a diagram illustrating a Modulation Transfer Function (MTF) curve.

### DETAILED DESCRIPTION OF THE INVENTION

Hereinafter, a medicine verification device and a medicine verification method of the present invention will be described in detail.

Although the description of the components described below might be made based on typical embodiments of the present invention, the present invention is not limited to such embodiments. That is, the following embodiments are an example provided to facilitate understanding of the medicine verification device and the medicine verification method of the present invention and would not limit the present invention. Accordingly, various improvements or alterations may be made without departing from the scope and spirit of the present invention.

In addition, in the present description, a “medicine” represents a solid medicine, and specifically corresponds to a tablet or a capsule.

<<Medicine Prescription Operation>>

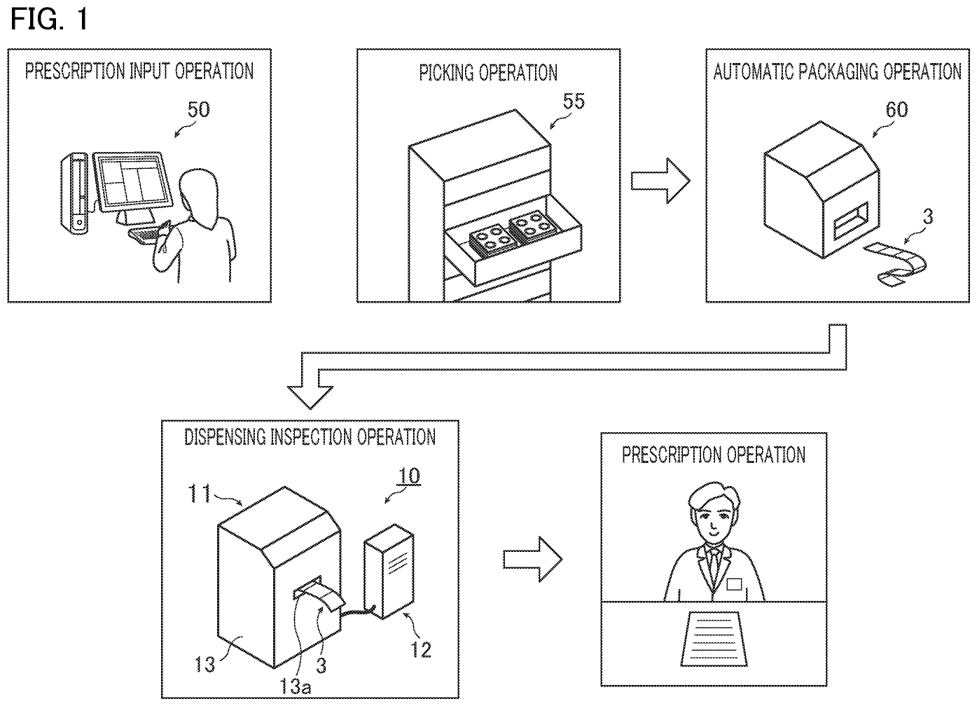

Before describing the medicine verification device (hereinafter, a medicine verification device 10) according to one embodiment of the present invention, a medicine prescription operation performed using the medicine verification device 10 will be outlined first. The medicine prescription operation includes operations sequentially performed in the order of a prescription input operation, a picking operation, an automatic packaging operation, a dispensing inspection operation, and a prescription operation, as illustrated in FIG. 1. FIG. 1 is a diagram illustrating a flow of the medicine prescription operation.

In the prescription input operation, a pharmacist inputs prescription conditions described in a prescription to a computer (hereinafter, a prescription condition input device 50). Here, a prescription condition is a condition set for prescribing a medicine to a patient. Examples of input prescription conditions include the name and age of the patient, the type of medicine to be prescribed, and the prescription quantity for each of types. The following description assumes that medicines are taken by a plurality of doses and that the prescription quantity for one dose is the same. However, the present invention is not limited to this, and a medicine for only one dose may be prescribed. Furthermore, the type and the prescription quantity of the medicine for one dose may be different each of times.

In the picking operation, on the basis of prescription conditions, a pharmacist picks medicines of the types corresponding to the prescription conditions from a medicine shelf 55 by the quantities according to the prescription conditions. Note that the picking operation is not limited to the case where the pharmacist performs the operation manually, but may be performed automatically by a known automatic picking device on the basis of the prescription conditions input to the prescription condition input device 50.

Furthermore, each of the medicines picked in the present embodiment includes identification information formed on a medicine surface. The “identification information” includes characters, numerals, symbols, or the like for identifying the type of medicine (medicine type) and is formed by engraving or printing. In the present embodiment, it is assumed that identification information is formed on the surface of the medicine by engraving (recess processing). However, the present invention is not limited to the above-described embodiment, and medicines to be picked may include medicines for which identification information is not formed or may include medicines for which identification information is formed by printing.

In the automatic packaging operation, the pharmacist sets the medicines picked in the picking operation onto a tray of a packaging machine 60 illustrated in FIG. 1, and then, the packaging machine 60 automatically packages the medicines in the tray. At this time, the picked medicines are set on a tray for one dose, and the medicines for one dose are packaged in each of the plurality of packaging bags 1. The packaging bag 1 is a bag-shaped sachet and is formed of a packaging material having light transmissivity. Examples of the material of the packaging bag 1 include a laminated film of cellophane and polyethylene, and a polyethylene film.

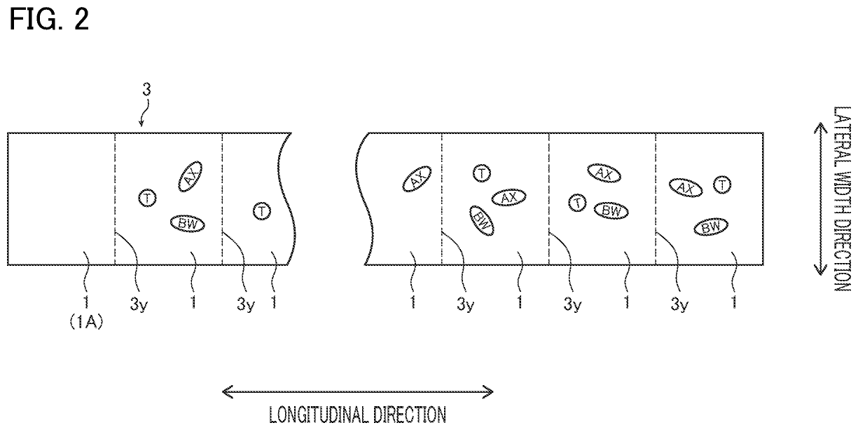

At a point of completion of the automatic packaging operation, the plurality of packaging bags 1 each containing the medicines are continuously arranged to form a continuous packaging bag 3 having a strip-like shape as illustrated in FIG. 2. At the time of taking the medicine, one packaging bag 1 is separated from the continuous packaging bag 3 and the medicine packed in the separated packaging bag 1 is taken. FIG. 2 is a view illustrating the continuous packaging bag 3.

Note that the plurality of packaging bags 1 are not limited to the form of being continuous as the continuous packaging bag 3 at a point of completion of the automatic packaging operation and may be separated from each other.

At a point of completion of the automatic packaging operation, the packaging bag 1 located at one end of the continuous packaging bag 3 is an empty packaging bag 1 containing no medicine (hereinafter, referred to as an empty bag 1A) as illustrated in FIG. 2. The empty bag 1A is similar to the packaging bag 1 packing the medicine, except for not containing the medicine inside. The empty bag 1A may be provided at a location other than the end of the continuous packaging bag 3. Moreover, there is no need to include the empty bag 1A in the continuous packaging bag 3.

In the dispensing inspection operation, inspection of whether the prescribed medicine is correct is performed using the medicine verification device 10 illustrated in FIG. 1. Specifically, inspection of whether the type and the number (more precisely, the number of each of types) of the medicines contained in each of the packaging bags 1 in the continuous packaging bag 3 are as specified in the prescription is performed.

The prescription operation performs prescription of prepackaged medicines determined to be correct (as instructed by the prescription) in the dispensing inspection operation, for the patient (prescription destination). At this time, the pharmacist removes the empty bag 1A located at one end of the continuous packaging bag 3 and hands the remaining continuous packaging bag 3 to the patient.

<<Configuration of Medicine Verification Device>>

Next, a configuration of the medicine verification device 10 will be described.

The medicine verification device 10 is used for dispensing inspection and verifies the type and the number (more precisely, the number of each type) of the medicine packed in the packaging bag 1 in the automatic packaging operation. Here, the medicine packed (packaged) in one packaging bag 1 corresponds to the “verification target medicine” of the present invention.

As illustrated in FIG. 1, the medicine verification device 10 includes: a device main body 11 having a function of capturing an image of a medicine as a verification target (specifically, a medicine packed in each of the packaging bags 1); and a processing device 12 having a function of performing dispensing inspection based on the image captured by the device main body 11.

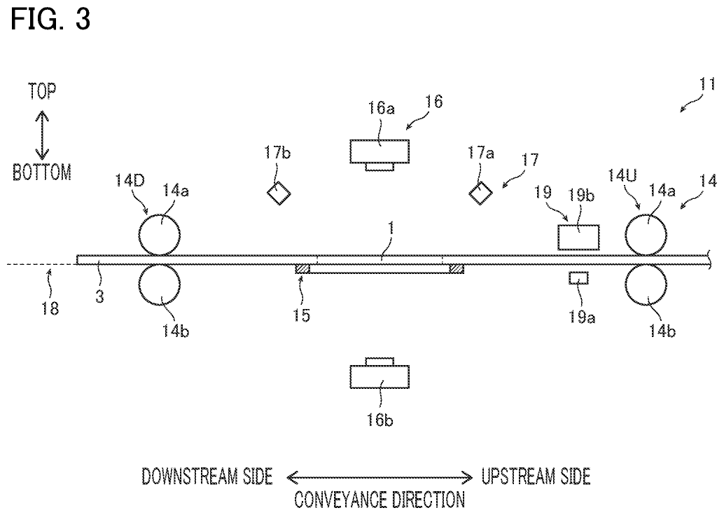

The device main body 11 includes a housing 13 illustrated in FIG. 1, and includes, within the housing 13, a conveyance unit 14, an arrangement unit 15, an image capturing unit 16, and a light irradiation unit 17 illustrated in FIG. 3. FIG. 3 is a schematic view illustrating an internal structure of the device main body 11. In addition, the housing 13 of the device main body 11 includes: an introduction part 13a for introducing the continuous packaging bag 3 to the inside of the device main body 11; and a discharge part (not illustrated) for discharging the continuous packaging bag 3 introduced inside the device main body 11 to the outside of the device main body 11.

The conveyance unit 14 has a conveyance path 18 formed inside the device main body 11 and conveys the continuous packaging bag 3 along the conveyance path 18. The continuous packaging bag 3 introduced into the inside of the device main body 11 from the introduction part 13a moves toward the downstream side of the conveyance path 18 by the conveyance operation of the conveyance unit 14 and eventually passes through the discharge part to be discharged to the outside of the device main body 11. Here, the “downstream side” means a side closer to the discharge part in the conveyance direction, and an “upstream side” means a side opposite to the downstream side, that is, a side closer to the introduction part 13a in the conveyance direction.

In the present embodiment, the conveyance path 18 is a horizontal path, and the conveyance unit 14 performs conveyance in a state where a longitudinal direction of the continuous packaging bag 3 runs along the conveyance path 18 (that is, the conveyance direction) and where a thickness direction of the continuous packaging bag 3 runs along the up-down direction (vertical direction).

As illustrated in FIG. 3, the conveyance unit 14 includes an upstream drive unit 14U and a downstream drive unit 14D. The upstream drive unit 14U is arranged on the upstream side of the arrangement unit 15, while the downstream drive unit 14D is arranged on the downstream side of the arrangement unit 15. Each of the upstream drive unit 14U and the downstream drive unit 14D includes a pair of upper and lower nip rollers 14a and 14b, and a motor (not illustrated) that rotationally drives one of the pair of upper and lower nip rollers 14a and 14b. The pair of upper and lower nip rollers 14a and 14b is arranged with a gap enough to allow the continuous packaging bag 3 to pass through, and the rollers rotate in a state of nipping the continuous packaging bag 3 between the rollers. With this configuration, the continuous packaging bag 3 is conveyed in a state where a slight tension is applied.

In the present embodiment, the motor is configured to rotate intermittently. Therefore, the conveyance unit 14 performs the conveyance operation intermittently. In one conveyance operation, the continuous packaging bag 3 moves by a predetermined amount in the conveyance direction. The movement amount (conveyance amount) of the continuous packaging bag 3 in one conveyance operation is set by the control unit 21 of the processing device 12 described below.

Note that the conveyance unit 14 of the present embodiment can perform conveyance to either the upstream side or the downstream side in the conveyance direction by switching the rotational direction of the motor. The conveyance direction is set by the control unit 21 of the processing device 12.

Furthermore, while the present embodiment is an example that applies the conveyance mechanism using the rotation drive of the roller (that is, a roller conveyor), it is also allowable to use other conveyance mechanisms as long as the mechanism can convey the continuous packaging bag 3 properly. For example, it is allowable to apply a belt conveyor that conveys the continuous packaging bag 3 by rotating an endless belt while the continuous packaging bag 3 is mounted on the upper surface of the belt.

The arrangement unit 15 is a portion where the verification target medicine is disposed in a state of being packed in the packaging bag 1 and is provided at an intermediate position of the conveyance path 18 as illustrated in FIG. 3. The arrangement unit 15 is a rectangular frame-shaped base and has a size on which one packaging bag 1 can be mounted. In addition, the packaging bags 1 disposed on the arrangement unit 15 in the continuous packaging bag 3 are sequentially switched together with the conveyance of the continuous packaging bag 3 by the conveyance unit 14.

Note that, in a state where the packaging bag 1 is disposed on the arrangement unit 15, the entire region of the upper surface of the packaging bag 1 (the surface facing the upper side of the device main body 11, the similar applies hereinafter) is exposed, while regions other than the edge of the lower surface of the packaging bag 1 (the surface facing the upper side of the device main body 11, the similar applies hereinafter) are exposed. The edge of the packaging bag 1 is a sealed portion formed by stacking and pressing two film sheets constituting the packaging bag 1 together.

The image capturing unit 16 captures, for each of the packaging bags 1, an image of the medicine packed in each of the packaging bags 1 in the continuous packaging bag 3, that is, an image of the verification target medicine, at an intermediate position of the conveyance path 18. More specifically, the image capturing unit 16 captures an image of the packaging bag 1 disposed on the arrangement unit 15 and an image of the medicine inside the packaging bag 1 every time the packaging bag 1 disposed on the arrangement unit 15 is switched.

As illustrated in FIG. 3, the image capturing unit 16 includes two cameras, upper and lower, as a plurality of cameras. The camera on the upper side (hereinafter, referred to as a first camera 16a) is disposed immediately above the arrangement unit 15 and captures an image of the medicine packed in the packaging bag 1 disposed on the arrangement unit 15 from above. The lower camera (hereinafter, referred to as a second camera 16b) is disposed immediately below the arrangement unit 15 and captures an image of the medicine packed in the packaging bag 1 disposed on the arrangement unit 15 from below. Here, the “medicine image” or “image of a medicine” means an image of the medicines captured through the packaging bag 1 in the present embodiment.

In the present embodiment, the conveyance operation by the conveyance unit 14 is to be performed intermittently, and the image capturing unit 16 captures an image of the packaging bag 1 disposed on the arrangement unit 15 and an image of a medicine packed in the packaging bag 1 between the conveyance operations. In addition, every time the packaging bag 1 disposed on the arrangement unit 15 is switched by the conveyance operation, the image capturing unit 16 captures the image of the packaging bag 1 disposed on the arrangement unit 15 and the image of the medicine packed in the packaging bag 1.

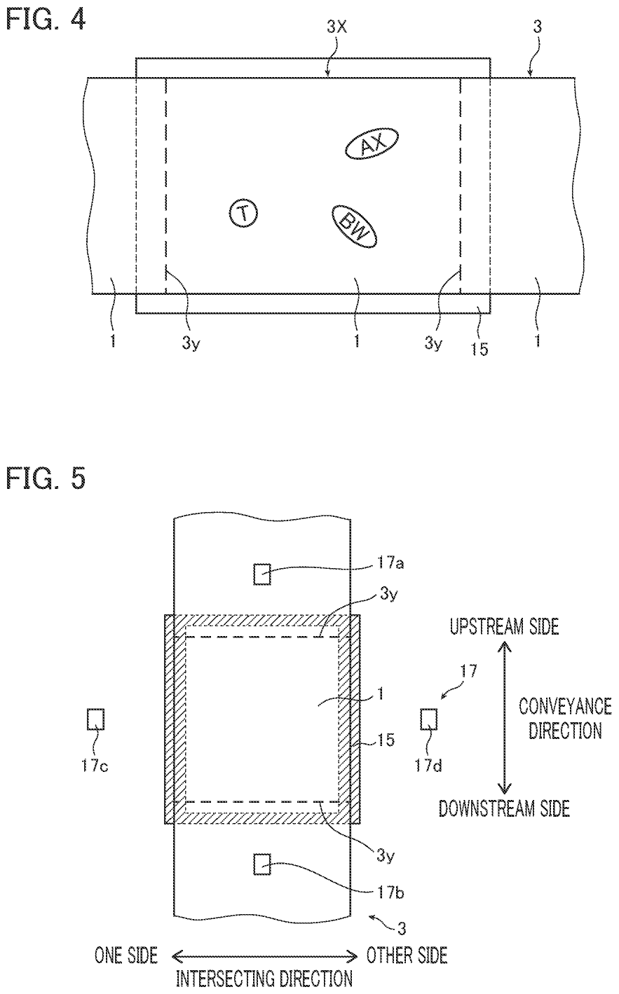

In the present embodiment, the imaging range of the first camera 16a is set to a rectangular region as illustrated in FIG. 4 (a rectangular region illustrated by a broken line in FIG. 4), which is a range capable of imaging an entire surface of the upper surface of the packaging bag 1 disposed on the arrangement unit 15 and a part (more precisely, an end portion) of the upper surface of the packaging bag 1 located on both sides of the packing bag 1 disposed on the arrangement unit 15. In other words, the portion of the continuous packaging bag 3 that is within the imaging range of the first camera 16a corresponds to an imaging target portion 3x, and at least the cutout line 3y between the packaging bags 3 is located at this portion as illustrated in FIG. 4. Here, the cutout line 3y is a boundary recess formed at a boundary position between the packaging bags 3 in the continuous packaging bag 3, and more specifically, is constituted by a dashed linear groove being formed from one end to the other end of the continuous packaging bag 3 in the lateral width direction of the continuous packaging bag 3.

FIG. 4 is a view illustrating an imaging range of the image capturing unit 16 and a part of the continuous packaging bag 3 included in the range.

Similarly, the imaging range of the lower camera 16b is set to a rectangular region, which is a range capable of imaging a region on the lower surface of the packaging bag 1 disposed on the arrangement unit 15 that is inside the arrangement unit 15 and is exposed, and a portion (more precisely, an end portion) of the lower surface of the packaging bag 1 located on both sides of the packing bag 1 disposed on the arrangement unit 15. In other words, the portion of the continuous packaging bag 3 that is within the imaging range of the second camera 16b corresponds to the imaging target portion 3x, and this portion includes at least the cutout line 3y between the packaging bags 1.

The image capturing unit 16 may be any type as long as it has a function of acquiring image data of a subject. Examples of this include a Charge-Coupled Device (CCD) image sensor and a Complementary Metal Oxide Semiconductor (CMOS) image sensor, although the present invention is not limited to these.

Furthermore, the image capturing unit 16 in the present embodiment is implemented by two cameras, but the number of cameras is not particularly limited and may be one, or three or more.

Furthermore, in the present embodiment, the camera is installed at a position vertically sandwiching the arrangement unit 15. However, the installation position of the camera can be set to any position as long as the packaging bag 1 disposed on the arrangement unit 15 and the medicine packed in the packaging bag 1 can be imaged satisfactorily.

The light irradiation unit 17 is configured to perform light emission to the packaging bag 1 disposed on the arrangement unit 15 and the medicine packed in the packaging bag 1 (that is, the verification target medicine) when the image capturing unit 16 captures an image. More specifically, when the image capturing unit 16 captures an image, the light irradiation unit 17 emits light toward the packaging bag 1 within an imaging range of the image capturing unit 16 of the continuous packaging bag 3 (more precisely, the surface of the imaging target portion 3x).

As illustrated in FIG. 5, the light irradiation unit 17 has a plurality of light emitting units, specifically four light emitting units 17a, 17b, 17c, and 17d in the present embodiment. FIG. 5 is a schematic top view of a plurality of light emitting units of the light irradiation unit 17. The four light emitting units 17a, 17b, 17c, and 17d are light sources used when the light irradiation unit 17 performs light emission, and individually arranged on four sides of the arrangement unit 15 as illustrated in FIG. 5. The light irradiation unit 17 performs light emission in different directions using the four light emitting units 17a, 17b, 17c, and 17d (a plurality of light emitting units).

More specifically, the two light emitting units 17a and 17b are arranged at positions opposite to each other as viewed from the arrangement unit 15 in the conveyance direction, and the units emit light in mutually opposite directions. That is, one light emitting unit 17a (hereinafter, referred to as a first light emitting unit 17a) emits light from the upstream side in the conveyance direction to the arrangement unit 15 located downstream in the conveyance direction. The other light emitting unit 17b (hereinafter, referred to as a second light emitting unit 17b) emits light from the downstream side in the conveyance direction to the arrangement unit 15 located upstream in the conveyance direction.

The remaining two light emitting units 17c and 17d out of the four light emitting units 17a, 17b, 17c, and 17d are arranged at positions opposite to each other as viewed from the arrangement unit 15 in a direction (hereinafter, an intersecting direction) intersecting the conveyance direction, and the units emit light in mutually opposite directions. That is, one light emitting unit 17c (hereinafter, referred to as a third light emitting unit 17c) emits light from one side in the intersecting direction toward the arrangement unit 15 on the other side. The other light emitting unit 17d (hereinafter, referred to as a fourth light emitting unit 17d) emits light to the arrangement unit 15 on one side, from the other side, in the intersecting direction. Here, “one side in the intersecting direction” means, for example, a side near one end of the arrangement unit 15 in the intersecting direction, while “the other side in the intersecting direction” means a side close to the other end of the arrangement unit 15 in the intersecting direction.

The light irradiation unit 17 uses a part or all of the four light emitting units 17a, 17b, 17c, and 17d to perform light emission to the packaging bag 1 disposed on the arrangement unit 15 and the medicine packed in the packaging bag 1. At this time, as observed from FIGS. 3 and 5, the light irradiation unit 17 performs light emission diagonally to the packaging bag 1 disposed on the arrangement unit 15 and the medicine (that is, the imaging target portion 3x) packed in the packaging bag 1. This direction is advantageous because applying light diagonally to the surface of the medicine can emphasize the contour of the identification information formed on the surface of the medicine (in particular, an edge portion of the contour at the object of light emission).

In addition, in the present embodiment, it is possible to switch the light emitting units 17a, 17b, 17c, and 17d used by the light irradiation unit 17 when the image capturing unit 16 captures an image. Specifically, the light irradiation unit 17 performs light emission using one of the four light emitting units 17a, 17b, 17c, and 17d. While the light irradiation unit 17 performs light emission from one light emitting unit, the image capturing unit 16 captures an image of the medicine packed in the packaging bag 1 disposed on the arrangement unit 15, by one imaging. Thereafter, the light irradiation unit 17 switches one light emitting unit used immediately before to another light emitting unit among the light emitting units 17a, 17b, 17c, and 17d and then, performs light emission using the light emitting unit 17a, 17b, 17c, or 17d after the switching. In the meantime, the image capturing unit 16 captures an image of the medicine packed in the packaging bag 1 disposed on the arrangement unit 15 again.

Thereafter, the light irradiation unit 17 sequentially switches the light emitting units 17a, 17b, 17c, and 17d using the similar procedure, and the image capturing unit 16 captures an image of the medicine packed in the packaging bag 1 disposed on the arrangement unit 15 every time the light irradiation unit 17 switches the light emitting units 17a, 17b, 17c, and 17d. This results in acquisition of captured images for each of the light irradiation directions (that is, four images having mutually different reflection states of light at each of portions of the medicine surface) for the medicine packed in one packaging bag 1 disposed on the arrangement unit 15. However, the present invention is not limited to this, and it is allowable to use a configuration in which two to four of the four light emitting units 17a, 17b, 17c, and 17d are simultaneously turned on, and the light irradiation unit 17 performs light emission toward the medicine packed in one packaging bag 1 disposed on the arrangement unit 15 simultaneously from two to four directions.

The light emitting units 17a, 17b, 17c, and 17d used by the light irradiation unit 17 for light emission may be implemented with known light sources, including any of a point light source, a line light source, or a surface light source. Specifically, examples of applicable light sources include: electroluminescence types such as Light Emitting Diode (LED), semiconductor laser (Laser Diode (LD)), and organic Electroluminescence (EL), radiant heat types such as halogen bulbs and incandescent bulbs, discharge emission types such as a mercury lamp and a fluorescent lamp, and a combination of these light sources with a light guide member such as a light guide plate or an optical fiber.

Furthermore, the present embodiment uses the light irradiation unit 17 having four light emitting units 17a, 17b, 17c, and 17d. However, the number of light emitting units (light sources) is not particularly limited, and it is sufficient as long as two or more units are provided.

Moreover, instead of arranging the four light emitting units 17a, 17b, 17c, and 17d around the arrangement unit 15, it is allowable to dispose one annular light emitting unit. With such a configuration, it is possible to irradiate the arrangement unit 15 with light from all directions of 360 degrees.

In addition to the parts described above (specifically, the conveyance unit 14, the arrangement unit 15, the image capturing unit 16, and the light irradiation unit 17), the device main body 11 also includes a measurement unit 19 illustrated in FIG. 3. The measurement unit 19 measures an optical characteristic of the packaging bag 1 and is provided inside the device main body 11 of the medicine verification device 10 in the present embodiment. Here, the “optical characteristic of the packaging bag 1” refers to an optical characteristic of the packaging bag 1 that influences the sharpness (in other words, the degree of blurring) of the image of the medicine packed in the packaging bag 1 captured by the image capturing unit 16, and more specifically refers to the light transmissivity and light scattering characteristics of the packaging bag 1. The measurement unit 19 according to the present embodiment measures the light transmittance of the packaging bag 1. However, the present invention is not limited to this, and a haze value (haze) may be measured as an optical characteristic of the packaging bag 1.

The measurement unit 19 has a configuration similar to a known light transmittance meter and includes a light source 19a and a light receiver 19b as illustrated in FIG. 3. The light source 19a and the light receiver 19b are arranged upstream of the arrangement unit 15 in the conveyance direction. A part of the conveyance path 18 is interposed between the light source 19a and the light receiver 19b in the up-down direction. Therefore, each of portions in the continuous packaging bag 3 (that is, each of the packaging bags 1) passes between the light source 19a and the light receiver 19b when being conveyed by the conveyance unit 14. The measurement unit 19 emits, from the light source 19a, light of a specific wavelength toward the packaging bag 1 disposed between the light source 19a and the light receiver 19b and receives the light transmitted through the packaging bag 1 by the light receiver 19b. Subsequently, the measurement unit 19 obtains the light transmittance based on the intensity of the light emitted from the light source 19a and the intensity of the light received by the light receiver 19b. The wavelength of the light emitted from the light source 19a can be set arbitrarily.

Furthermore, in the present embodiment, the measurement unit 19 measures an optical characteristic, more precisely, the light transmittance, of the packaging bag 1 for at least one packaging bag 1 of the continuous packaging bag 3 at an intermediate position of the conveyance path 18. More specifically, in the present embodiment, the measurement unit 19 measures the light transmittance of the empty packaging bag 1 containing no medicine (that is, the empty bag 1A) of the continuous packaging bag 3. This makes it possible to measure the light transmittance of the packaging bag 1 with higher accuracy. However, the present invention is not limited to this, and the light transmittance may be measured for the packaging bag 1 containing a medicine.

Furthermore, in the present embodiment, the measurement unit 19 measures the light transmittance of the empty bag 1A present in the continuous packaging bag 3 every time a new continuous packaging bag 3 is introduced into the device main body 11.

The processing device 12 executes a series of information processing in the implementation of the dispensing inspection. In the present embodiment, the processing device 12 is constituted using a personal computer (PC) external to the device main body 11. However, the present invention is not limited to this, and the processing device 12 may be constituted using a computer built in the device main body 11.

Furthermore, the processing device 12 is communicably connected to the device main body 11, the prescription condition input device 50, and a server computer 70 described below. The connection method between the processing device 12 and each of devices may be a wired connection method or a wireless connection method.

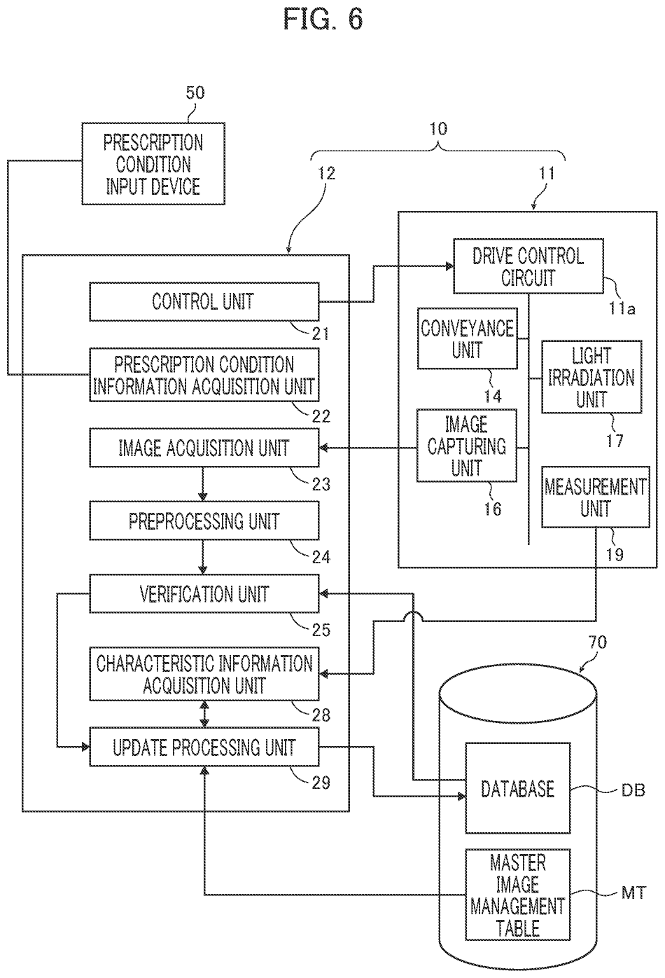

Furthermore, as illustrated in FIG. 6, the processing device 12 includes a control unit 21, a prescription condition information acquisition unit 22, an image acquisition unit 23, a preprocessing unit 24, a verification unit 25, a characteristic information acquisition unit 28, and an update processing unit 29. FIG. 6 is a block diagram illustrating a configuration of the processing device 12. These individual units are implemented by cooperation of hardware devices such as a Central Processing Unit (CPU) and a memory (not illustrated) included in the processing device 12, and an information processing program stored in the processing device 12. The information processing program may be read and obtained from a recording medium such as a Compact Disc Read Only Memory (CD-ROM) storing the program, or may be downloaded and obtained from a predetermined site via a network.

In the present embodiment, individual functional units of the processing device 12 (specifically, the control unit 21, the prescription condition information acquisition unit 22, the image acquisition unit 23, the preprocessing unit 24, the verification unit 25, the characteristic information acquisition unit 28, and the update processing unit 29) are constituted by one personal computer. However, the present invention is not limited to this, and it is allowable to use a configuration in which a part of the above functional units is constituted by one personal computer while the remaining functional units are constituted by another personal computer.

The control unit 21 is electrically connected to each of units of the device main body 11 (specifically, the conveyance unit 14, the image capturing unit 16, the light irradiation unit 17, and the measurement unit 19) via a drive control circuit 11a mounted on the device main body 11 and controls each of the units of the device.

More specifically, the control unit 21 performs control related to the conveyance operation of the conveyance unit 14, such as controlling a conveyance amount, a conveyance direction, a conveyance operation timing, or the like in one conveyance operation. In addition, the control unit 21 performs control related to the imaging operation of the image capturing unit 16, such as controlling a camera to be used among the two cameras 16a and 16b of the image capturing unit 16, and a timing of imaging. In addition, the control unit 21 performs control related to the light emitting operation of the light irradiation unit 17, such as controlling the light emitting unit to be used among the four light emitting units 17a, 17b, 17c, and 17d included in the light irradiation unit 17, and the timing of light emission.

Furthermore, the control unit 21 controls the measurement timing (measurement start point) regarding the light transmittance measurement performed by the measurement unit 19. Specifically, at a time when the continuous packaging bag 3 is introduced into the device main body 11 and the leading end of the continuous packaging bag 3 (the end located on the most downstream side in the conveyance direction) passes through a position immediately before a space between the light source 19a and the light receiver 19b of the measurement unit 19 in the conveyance path 18, the control unit 21 turns on the light source 19a and causes the measurement unit 19 to start light transmittance measurement.

The prescription condition information acquisition unit 22 is communicably connected to the prescription condition input device 50 and acquires prescription condition information by communicating with the prescription condition input device 50. Here, the prescription condition information is information indicating the prescription condition, which specifically is electronic data indicating the prescription condition input to the prescription condition input device 50 by the pharmacist.

In the present embodiment, when the input of the prescription condition is completed in the prescription condition input device 50, the prescription condition information is automatically transmitted from the prescription condition input device 50 to the prescription condition information acquisition unit 22, and then the prescription condition information acquisition unit 22 receives the above prescription condition information. However, the present invention is not limited to this. It is also allowable to use a configuration in which an information transmission request is transmitted from the prescription condition information acquisition unit 22, and the prescription condition input device 50 transmits the prescription condition information at a point of reception of the request by the prescription condition input device 50. More specifically, character string information or the two-dimensional barcode information for specifying the prescription condition is printed on a tip portion of the continuous packaging bag 3 (a portion of the continuous packaging bag 3 first introduced into the device main body 11). The prescription condition information acquisition unit 22 reads the above-described printed information when the continuous packaging bag 3 is introduced into the device main body 11. Thereafter, based on the read printed information, the prescription condition information acquisition unit 22 requests a prescription condition information indicating prescription conditions related to the medicine packaged in each of the packaging bags 1 of the continuous packaging bag 3 introduced into the device main body 11, against the prescription condition input device 50. After receiving this request, the prescription condition input device 50 analyzes the request, specifies prescription condition information related to the request, and transmits the specified prescription condition information to the processing device 12.

The image acquisition unit 23 is connected to the image capturing unit 16 (more precisely, the first camera 16a and the second camera 16b) and acquires, via a network, an image captured by the image capturing unit 16. Here, the image acquired by the image acquisition unit 23 corresponds to image data in specifically, Joint Photographic Experts Group (JPEG) format, Graphics Interchange Format (GIF) format, Portable Network Graphics (PNG) format, Tagged Image File Format (TIFF) format, Bitmap Image (BMP) format, or the like.

Note that the image acquisition unit 23 acquires an image from the image capturing unit 16 each time the image capturing unit 16 captures an image. More specifically, in the present embodiment, as described above, images are captured a plurality of times (specifically, eight times) separately for each of imaging conditions for one packaging bag 1 in which medicines are packaged. Accordingly, the image acquisition unit 23 acquires images for various imaging conditions (that is, eight images) for each of the packaging bags 1 and the medicines packed in the packaging bags 1. Furthermore, when the packaging bag 1 disposed on the arrangement unit 15 is switched, the image capturing unit 16 newly captures an image for each of imaging conditions. Accordingly, the image acquisition unit 23 acquires the newly captured image for each of the imaging conditions.

The preprocessing unit 24 performs preprocessing on the image acquired by the image acquisition unit 23 from the image capturing unit 16 (that is, the image data of the verification target medicine). The preprocessing is a process for emphasizing the identification information formed on the surface of the medicine appearing in the image acquired by the image acquisition unit 23.

More specifically, in the present embodiment, as described above, an image is captured a plurality of times (specifically, four times) in various light irradiation directions for the medicine packed in one packaging bag 1. Here, each of the images for each of the light irradiation directions has unevenness in light illuminance occurring on the surface of the medicine appearing in the image. Such uneven illuminance of light has an influence in detecting and specifying identification information formed on the surface of the medicine. Furthermore, the illuminance unevenness of light varies depending on the light irradiation direction. Therefore, the preprocessing unit 24 performs preprocessing. For an image captured for each of light irradiation directions, the preprocessing uses an edge extraction filter in a direction corresponding to the irradiation direction, which is an edge extraction filter of a size corresponding to the number of pixels of an edge (marked groove) of the identification information appearing in each of the images, thereby generating an edge image for each of irradiation directions, and thereafter, generates a combined image in which a plurality of edge images are combined. The edge extraction filter can include at least one of a Sobel filter, a Laplacian filter, and a Canny filter, and can be appropriately selected according to a verification method to be described below.

The image that has undergone the above preprocessing is an image from which the illuminance unevenness of light that varies according to the light irradiation direction is eliminated as much as possible and in which the identification information formed on the surface of the medicine appearing in the image is emphasized. Specifically, it is possible to reduce the information other than the engraving, such as the pattern and the scratches smaller than the groove of engraving indicating the identification information on the surface of the medicine, leading to extraction of the engraving.

The verification unit 25 verifies the type and the number of medicines (that is, verification target medicines) packed in the packaging bag 1 disposed on the arrangement unit 15. More specifically, the verification unit 25 uses a master image corresponding to the type of medicine specified from the prescription condition information and an image of the verification target medicine captured by the image capturing unit 16 (more precisely, an image preprocessed by the preprocessing unit 24). Subsequently, the verification unit 25 uses these images to verify the type of verification target medicine and the number of medicines for each of types.

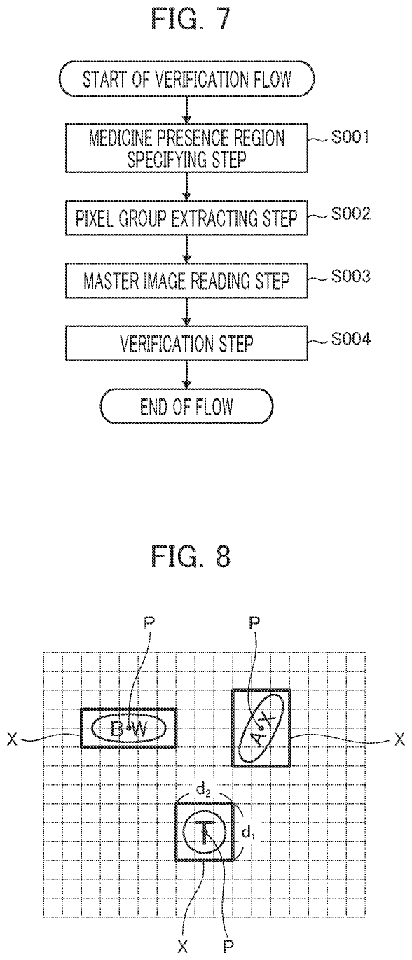

The specific procedure of the verification performed by the verification unit 25 will be described with reference to FIG. 7. FIG. 7 is a diagram illustrating a general procedure of a verification flow performed by the verification unit 25. The verification flow of the verification unit 25, as illustrated in FIG. 7, first performs a step of specifying a region including an image of a verification target medicine from the image on which preprocessing has been performed (S001). Hereinafter, an image on which preprocessing has been performed is referred to as a “preprocessed image”, and a region including an image of a medicine in the preprocessed image is referred to as a “medicine presence region”.

In the present embodiment, the preprocessing is performed first, and then a medicine presence region specifying step S001 and a pixel group extracting step S002 described below are performed on the preprocessed image obtained by the preprocessing. However, the present invention is not limited to this mode. It is allowable to use a mode in which the medicine presence region specifying step S001 and the pixel group extracting step S002 are performed on each of the captured images obtained for each of light irradiation directions, and then preprocessing is performed on the captured images for each of irradiation directions in which these steps were performed (more precisely, a medicine extraction image X described below).

The medicine presence region specifying step S001 performs a known edge extraction process and a known segmentation process on the preprocessed image to specify a contour of the medicine in the image. Subsequently, a region surrounded by the specified contour is specified as a medicine presence region. In a case where a plurality of medicines appear in the preprocessed image, the medicine presence regions are specified as many as the number of medicines.

After performing the medicine presence region specifying step S001, the verification unit 25 extracts a pixel group corresponding to the medicine presence region from among the pixel group forming the preprocessed image (S002). As illustrated in FIG. 8, the extracted pixel group forms a rectangle (rectangular region denoted by reference symbol X in FIG. 8) surrounding the medicine presence region. Hereinafter, the extracted pixel group is referred to as a “medicine extraction image X”. In a case where a plurality of medicine presence regions are specified in the medicine presence region specifying step S001, the medicine extraction image X is specified for each of medicine presence regions.

FIG. 8 is a view illustrating the medicine extraction image X. Note that the pixel size (single pixel size with respect to the image) illustrated in FIG. 8 is larger than the actual pixel size for the sake of convenience of illustration.

In the pixel group extracting step S002, the size and the position of the medicine extraction image X are specified. Here, the size of the medicine extraction image X is the area of a rectangular pixel group forming the medicine extraction image X and corresponds to the product of lengths d1 and d2 of the two sides illustrated in FIG. 8.

Furthermore, the position of the medicine extraction image X is a coordinate position when the reference position is an origin and the conveyance direction and the intersecting direction are defined as coordinate axis directions. Specifically, this corresponds to an intersecting position of the diagonal lines of the rectangular pixel group forming the medicine extraction image X, namely the coordinates of point P illustrated in FIG. 8. By specifying the position of the medicine extraction image X in this manner, it is possible to specify the imaging position (arrangement position) of the verification target medicine corresponding to the imaging range of the image capturing unit 16 (more precisely, the angle of view of each of the first camera 16a and the second camera 16b). While the present embodiment is a case where the reference position as the origin is set at the center position of the imaging range of the image capturing unit 16 (more precisely, the first camera 16a and the second camera 16b), the position is not limited to this and it may be set at any position.

After execution of the pixel group extracting step S002, the verification unit 25 executes a step of specifying the type of medicine to be prescribed from the prescription condition information acquired using the prescription condition information acquisition unit 22 and then reading a master image of the specified type of medicine from the database DB (S003). In this step S003, in a case where a plurality of types of medicines to be prescribed exist, that is, where a plurality of types of medicines are packaged in the packaging bag 1, a master image is read for each of types.

Here, the master image will be described. The master image is an image of a medicine registered corresponding to the type of medicine and is an image registered in advance for the type of medicine specified from the prescription condition information. Furthermore, in the present embodiment, the master image is obtained from an image of a medicine captured in a state of being packed in the packaging bag 1.

Note that there may be a case where a master image has not been registered for a new medicine or the like. For a medicine of a type for which a master image is not registered, a captured image obtained when the image capturing unit 16 first captures the medicine (more precisely, the medicine extraction image X) is to be registered as a master image. The following description will assume a case where master images are registered in advance for all the verification target medicines.

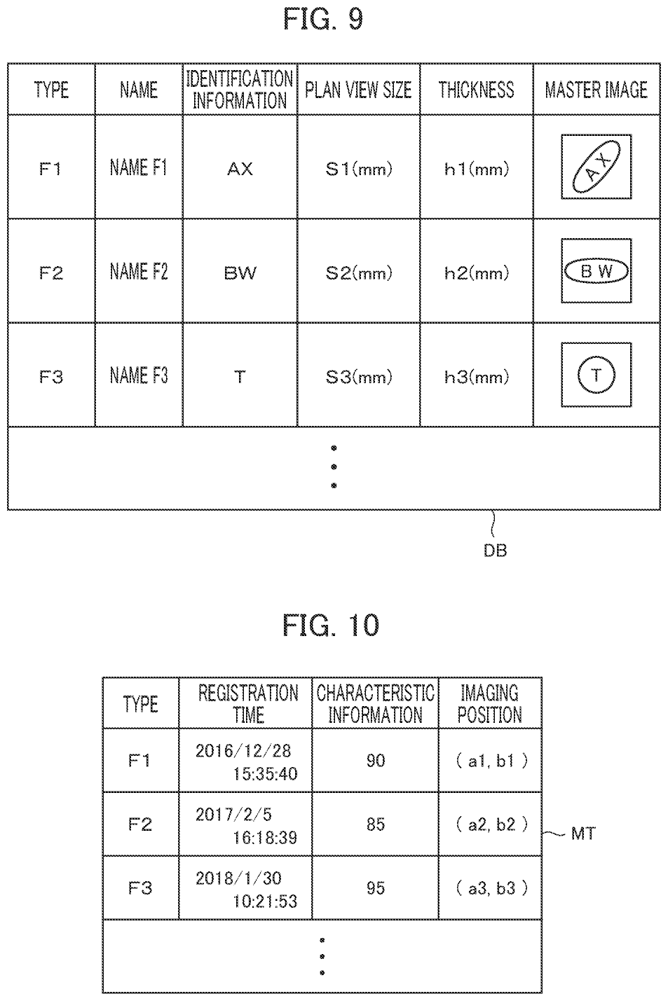

The database DB will be described. As illustrated in FIG. 9, the database DB is a database in which a master image of each of medicines and a type of the medicine are registered in association with each other. FIG. 9 is a diagram illustrating a database DB in which master images are registered.

In addition to the master image, the database DB includes a medicine name, identification information formed on the surface of the medicine, a plan view size and a thickness as medicine size individually registered in association with the type of the medicine. The information registered in the database DB is not limited to the above information, and information other than the above information may be registered.

In the present embodiment, the database DB is provided in a server computer 70 externally provided, and the verification unit 25 communicates with the server computer 70 to access the database DB. That is, in the present embodiment, the server computer 70 stores the master image. However, the present invention is not limited to this, and the database DB including the master image may be stored in the storage medium in the processing device 12.

After reading the master image from the database DB, the verification unit 25 verifies the type of the verification target medicine and the number of medicines for each of types (S004) using the read master image and the image of the verification target medicine captured by the image capturing unit 16 (more precisely, the medicine extraction image X).

More specifically, in the verification step S004, template matching with the master image is performed for each of the plurality of medicine extraction images X to evaluate the similarity (correlation value) with the master image. Examples of an applicable similarity evaluation method include a known geometric hashing method or a Locally Likely Arrangement Hashing (LLAH) method. Subsequently, it is verified that the type of medicine appearing in the image having the highest similarity among the plurality of medicine extraction images X matches the type of medicine appearing in the master image.

By repeating the above procedure for the read master images (that is, for the number of medicine types indicated by the prescription condition information acquired by the prescription condition information acquisition unit 22), the type is specified for each of verification target medicines. Thereafter, the verification unit 25 totals individually the number of medicines whose types are specified and counts the number of each of types.

The verification is performed by the verification unit 25 using the procedure described above. In addition, when the packaging bag 1 disposed on the arrangement unit 15 is switched (that is, when the verification target medicine changes), the verification is repeated each of times. That is, when the packaging bag 1 disposed on the arrangement unit 15 is switched and an image of the medicine packed in the packaging bag 1 is acquired, verification is performed using the newly acquired image. When the medicines are packaged under the same prescription condition in each of the packaging bags 1 of the continuous packaging bag 3, the master image used in the first verification can be used as it is in the second and subsequent verifications. Accordingly, step S003 of reading an image from the database DB may be omitted.

With the procedure described above, verification is performed on the medicine packed in each of the packaging bags 1 of the continuous packaging bag 3 (more precisely, the packaging bag 1 other than the empty bag 1A), and thus it is possible to inspect whether the medicine is correctly packaged in each of the packaging bags 1 as instructed by the prescription.

The characteristic information acquisition unit 28 acquires characteristic information. The characteristic information refers to information indicating an optical characteristic of the packaging bag 1 described above, specifically, information indicating at least one of the light transmissivity and the light scattering characteristic of the packaging bag 1, represented by the information indicating the light transmittance of the packaging bag 1 in the present embodiment. More specifically, the characteristic information acquisition unit 28 according to the present embodiment acquires the optical characteristic measured by the measurement unit 19, particularly, characteristic information indicating the measurement result of the light transmittance. That is, when the measurement unit 19 has measured the light transmittance of the packaging bag 1 for the empty bag 1A in the continuous packaging bag 3, the characteristic information acquisition unit 28 acquires, from the measurement unit 19, information (data) indicating the light transmittance of the packaging bag 1 measured by the measurement unit 19 through a transmission path (not illustrated) included in the measurement unit 19.

As described above, the characteristic information acquisition unit 28 acquires information indicating the light transmittance of the empty bag 1A in the continuous packaging bag 3, in which the light transmittance is the same between the empty bag 1A and other packaging bag 1 in the continuous packaging bag 3. Therefore, by acquiring the information indicating the light transmittance of the empty bag 1A in the continuous packaging bag 3, the characteristic information acquisition unit 28 acquires characteristic information (information indicating the light transmittance) for all the packaging bags 1 of the continuous packaging bag 3 introduced into the device main body 11. However, the present invention is not limited to this, and the measurement unit 19 may measure the light transmittance of each of the packaging bags 1 in the continuous packaging bag 3 one by one, and the characteristic information acquisition unit 28 may acquire the information indicating the light transmittance of the packaging bag 1 for each of the packaging bags 1.

Furthermore, when a new continuous packaging bag 3 is introduced into the device main body 11 of the medicine verification device 10 and the measurement unit 19 measures the light transmittance of the packaging bag 1 for the empty bag 1A in the continuous packaging bag 3, the characteristic information acquisition unit 28 acquires a measurement result of the measurement unit 19 every time the measurement is performed.

The update processing unit 29 executes update processing. The update processing is processing for updating the master image as an updating target among the master images registered in the database DB to the image (more precisely, the medicine extraction image X) of the verification target medicine verified by the verification unit 25 to have a type that matches the type of medicine appearing in the master image as the updating target.

In the update processing, the update processing unit 29 generates update request data and transmits this update request data to the server computer 70. The update request data includes data that specifies the master image as an update target, and an image of the verification target medicine to be the updated master image (that is, an image of the verification target medicine verified by the verification unit 25 to have a type that matches the type of medicine appearing in the master image as an update target). After receiving the update request data from the update processing unit 29, the server computer 70 specifies the master image as an update target from the database DB, and changes the master image to the image of the verification target medicine transmitted from the update processing unit 29.

In addition, after updating the master image, the server computer 70 updates a master image management table MT. The master image management table MT records information regarding the latest master image registered in the database DB, and records the type of medicine, registration time, characteristic information, and imaging position for each of the master images, as illustrated in FIG. 10. FIG. 10 is a diagram illustrating the master image management table MT.

Regarding the information recorded in the master image management table MT, the type of medicine is information indicating the type of medicine appearing in the master image. The registration time is information indicating the registration time or the latest update time for the master image. The characteristic information is the characteristic information of the packaging bag 1 appearing in the master image, more specifically, the characteristic information acquired by the characteristic information acquisition unit 28 for the packaging bag 1 packing the medicine appearing in the master image, and particularly corresponds to information indicating the light transmittance measured by the measurement unit 19 of the medicine verification device 10.

The imaging position is a position of the verification target medicine registered as the master image with respect to the image capturing unit 16 at a time when the image of the verification target medicine is captured by the image capturing unit 16. More specifically, the imaging position is a coordinate position when the reference position is the origin and the conveyance direction and the intersecting direction are the coordinate axis directions, and this indicates the center position of the medicine extraction image X registered as the master image, corresponding to the coordinates of point P illustrated in FIG. 8. In the present embodiment, the reference position being the origin is set at the center position of the imaging range (more precisely, the angle of view of each of the first camera 16a and the second camera 16b) of the image capturing unit 16. However, the present invention is not limited to this, and the reference position may be set to any position.

Note that the information recorded in the master image management table MT is not limited to the above-described information and may include information other than the above-described information. Furthermore, while the master image management table MT is stored in the server computer 70 in the present embodiment, the present invention is not limited to this. The master image management table MT may be stored in the storage medium of the processing device 12.

Returning to the description of the update processing unit 29. When the verification unit 25 has verified that the type of verification target medicine matches the type of medicine appearing in the master image (that is, the type of medicine specified from the prescription condition information), the update processing unit 29 determines the necessity of executing the update processing and executes the update processing in a case where it is determined that the execution of the update processing is necessary.

Specifically, in a stage previous to the verification of the suitability of the verification target medicine, the characteristic information acquisition unit 28 has acquired the characteristic information indicating the light transmittance of each of the packaging bags 1 of the continuous packaging bag 3.

Therefore, in a case where the verification unit 25 has verified that the type of the verification target medicine matches the type of the medicine appearing in the master image, the characteristic information acquisition unit 28 has acquired the information indicating the light transmittance of the packaging bag 1 packing the medicine for the verification target medicine verified by the verification unit 25 to have the type that matches the type of the medicine appearing in the master image. Hereinafter, for convenience of explanation, the verification target medicine verified by the verification unit 25 to have a type that matches the type of the medicine appearing in the master image is referred to as a “verified medicine”, and the packaging bag 1 packing the verified medicine is referred to as a “verified medicine packaging bag”.

The update processing unit 29 determines whether the optical characteristic indicated by the characteristic information acquired by the characteristic information acquisition unit 28 for the verified medicine packaging bag (that is, the light transmittance of the verified medicine packaging bag) satisfies the standard for the optical characteristic. Subsequently, when the light transmittance of the verified medicine packaging bag satisfies the standard, the update processing unit 29 executes the update processing of setting the image of the verified medicine as the master image.

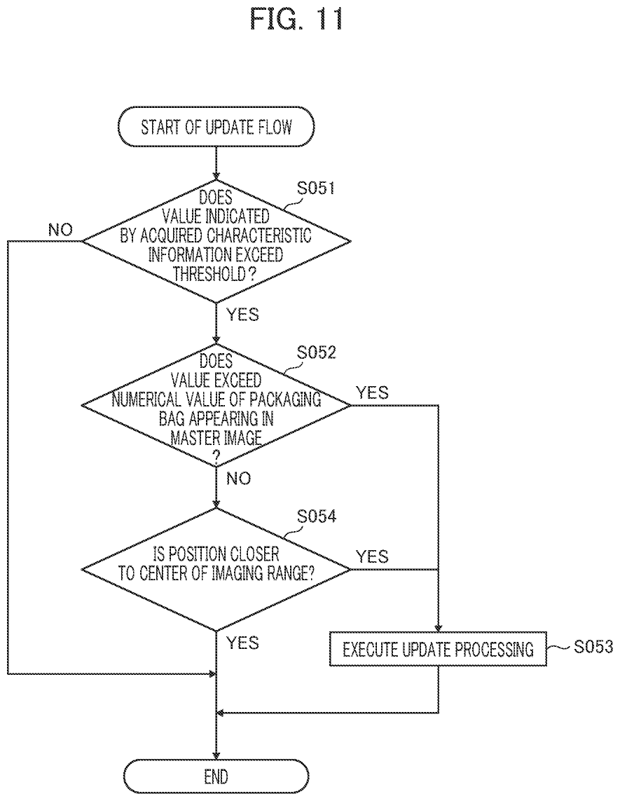

The determination of the necessity of executing the update processing will be described in detail. The update processing unit 29 determines the necessity of executing the update processing following the determination flow illustrated in FIG. 11 (hereinafter referred to as an update flow). FIG. 11 is a diagram illustrating an update flow. Hereinafter, how the determination is to be made by the update processing unit 29 as for the necessity of executing the update processing will be described with reference to FIG. 11.

The update processing unit 29 determines whether the optical characteristic (that is, light transmittance) indicated by the characteristic information of the verified medicine packaging bag acquired by the characteristic information acquisition unit 28 exceeds a threshold (S051). Here, the threshold is a value preset as a standard for determining whether to execute the update processing, which is stored in the storage medium of the processing device 12. The update processing unit 29 reads this threshold and specifies the magnitude relationship between the light transmittance of the verified medicine packaging bag and the threshold.

Subsequently, when the update processing unit 29 has determined that the light transmittance of the verified medicine packaging bag exceeds the threshold (Yes in S051), the update processing unit 29 further determines whether the light transmittance of the verified medicine packaging bag exceeds the light transmittance of the packaging bag 1 appearing in the master image (S052). More specifically, the update processing unit 29 communicates with the server computer 70 to access the master image management table MT stored in the server computer 70. The update processing unit 29 reads, from the master image management table MT, characteristic information of the master image corresponding to the type of the verified medicine (in other words, the type of medicine specified from the prescription condition information), and then specifies the optical characteristic indicated by the read characteristic information. For example, when the type of the verified medicine is F1 in the case of using the master image management table MT illustrated in FIG. 10, the optical characteristic (that is, light transmittance) indicated by the characteristic information is 90.

Subsequently, in a case where the update processing unit 29 has determined that the light transmittance of the verified medicine packaging bag exceeds the light transmittance of the packaging bag 1 appearing in the master image (Yes in S052), the update processing unit 29 executes the update processing of setting the image of the verified medicine as a master image (S053).

As described above, the update processing unit 29 determines whether the light transmittance of the verified medicine packaging bag acquired by the characteristic information acquisition unit 28 exceeds a threshold and whether the light transmittance exceeds the light transmittance of the packaging bag 1 appearing in the master image. Subsequently, the update processing unit 29 executes the update processing when the light transmittance of the verified medicine packaging bag exceeds the threshold and the light transmittance of the packaging bag 1 appearing in the master image. This makes it possible to update the master image so that a clearer image with a higher sharpness will be saved as the master image, enabling the subsequent determination (inspection) using the updated master image to be performed with higher accuracy.

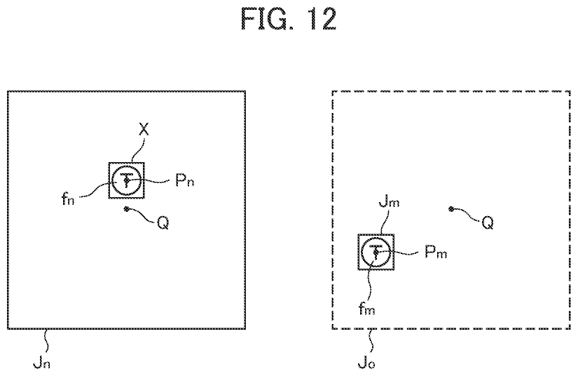

On the other hand, in a case where the update processing unit 29 has determined that the light transmittance of the verified medicine packaging bag does not exceed the light transmittance of the packaging bag 1 appearing in the master image (No in S052), the update processing unit 29 determines whether the imaging position of the verified medicine is closer to the center of the imaging range of the image capturing unit 16 than the imaging position of the medicine appearing in the master image (S054). The procedure of such determination will be specifically described by using an exemplary case illustrated in FIG. 12. FIG. 12 is a diagram illustrating a procedure for determining the necessity of executing the update processing, and more specifically, a diagram illustrating a specific example for describing the determination procedure in step S054 of the update flow.

Referring to FIG. 12, the left half of the figure illustrates a captured image Jn obtained when the image capturing unit 16 captures the imaging target portion 3x including a verified medicine fn in the continuous packaging bag 3. The right half of FIG. 12 illustrates a master image Jm used in the verification targeting the verified medicine and an extraction source image Jo from which the master image Jm has been extracted. The medicine appearing in the master image Jm (hereinafter, referred to as a master image medicine fm) corresponds to the past verified medicine. The extraction source image Jo corresponds to a captured image obtained when the image capturing unit 16 captures the imaging target portion 3x including the master image medicine fm in the continuous packaging bag 3.

The update processing unit 29 specifies the coordinate position (position indicated by symbol Pn in FIG. 12) at the center of the medicine extraction image X that is an image of the verified medicine fn, as the imaging position of the verified medicine fn. This position is the same as the position of the medicine extraction image X specified in the verification flow.

Furthermore, the update processing unit 29 specifies the coordinate position at the center of the master image Jm (position indicated by symbol Pm in FIG. 12), as the imaging position of the master image medicine fm. More specifically, the update processing unit 29 accesses the master image management table MT stored in the server computer 70, and reads the imaging position (refer to FIG. 10) of the master image corresponding to the type of the master image medicine fm, thereby specifying the coordinate position Pm at the center of the master image Jm.

After specifying the two coordinate positions Pm and Pn, the update processing unit 29 determines which coordinate position is closer to the center position (position indicated by symbol Q in FIG. 12) of the imaging range of the image capturing unit 16. In the case illustrated in FIG. 12, the update processing unit 29 is to determine that the coordinate position Pn, that is, the imaging position of the verified medicine fn is closer to the center position of the imaging range of the image capturing unit 16.

Subsequently, after determining that the imaging position of the verified medicine fn is closer to the center Q of the imaging range of the image capturing unit 16 than the imaging position of the master image medicine fm (Yes in S054), the update processing unit 29 executes update processing of setting the image of the verified medicine as the master image (S053).

As described above, the update processing unit 29 determines whether the light transmittance indicated by the characteristic information acquired by the characteristic information acquisition unit 28 for the verified medicine packaging bag exceeds a threshold, and determines whether the imaging position of the verified medicine is closer to the center Q of the imaging range of the image capturing unit 16 than the imaging position of the master image medicine. Subsequently, when the light transmittance of the verified medicine packaging bag exceeds the threshold and when the imaging position of the verified medicine is closer to the center Q of the imaging range of the image capturing unit 16 than the imaging position of the master image medicine, the update processing unit 29 executes update processing. The image of the medicine captured at a position closer to the center Q of the imaging range of the image capturing unit 16 is more similar to the front image of the medicine than the image of the medicine captured at a position farther from the center Q. Therefore, executing the above makes it possible to update the master image so that an image more similar to the front image will be saved as the master image, enabling subsequent determinations using the updated master image to be performed with higher accuracy.

In contrast, when the update processing unit 29 determines that the imaging position of the verified medicine fn is farther from the center Q of the imaging range of the image capturing unit 16 than the imaging position of the master image medicine fm (No in S054), the update processing unit 29 does not execute the update processing. In addition, when the update processing unit 29 determines that the light transmittance of the verified medicine packaging bag acquired by the characteristic information acquisition unit 28 is below the threshold (No in S051), the update processing unit 29 does not execute the update processing.

Although an exemplary procedure of the update flow has been described above, it is not limited to the above flow, the update processing may be executed at all times in a case, for example, where the light transmittance of the verified medicine packaging bag acquired by the characteristic information acquisition unit 28 exceeds a threshold. Furthermore, it is not necessary to use a threshold. In this case, it is allowable to determine whether the light transmittance of the verified medicine packaging bag exceeds the light transmittance of the packaging bag 1 appearing in the master image (that is, determination similar to step S052 described above is performed) so as to determine the necessity of executing the update processing.

Furthermore, regarding a new medicine for which no master image is registered, the captured image (more precisely, the medicine extraction image X) obtained when the image capturing unit 16 images the new medicine for the first time is to be registered as the master image as described above. Here, in a case where a plurality of captured images of a new medicine can be obtained, for example, where a plurality of new medicines of the same type is present in one packaging bag 1, it is desirable to register one captured image that satisfies the standard most as the master image.

<<Basic Operation of Medicine Verification Device>>

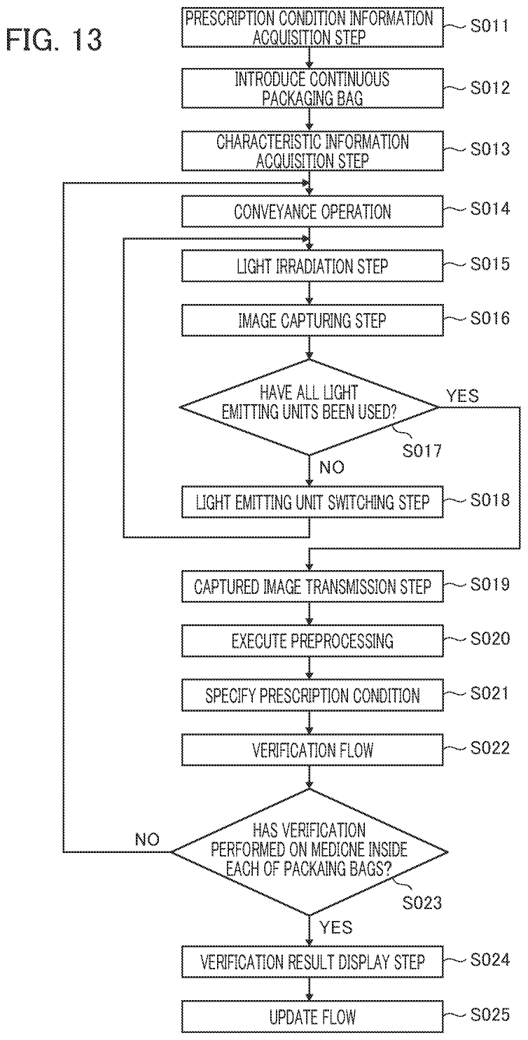

Next, basic operation of the medicine verification device 10 will be described with reference to FIG. 13. FIG. 13 is a diagram illustrating a flow of basic operation of the medicine verification device 10. In the basic operation of the medicine verification device 10 described below, the medicine verification method of the present invention is implemented, and in particular, prescription condition information acquisition S011, a characteristic information acquisition step S013, an image capturing step S016, a verification flow S022, and an update processing execution step S025 in the basic operation constitute the medicine verification method of the present invention.

First, after completion of the input of the prescription condition information in the prescription input operation, the prescription condition information acquisition unit 22 of the processing device 12 communicates with the prescription condition input device 50 and acquires the prescription condition information indicating the input prescription condition (S011).

Meanwhile, after the automatic packaging operation is performed by the packaging machine 60 in accordance with the input prescription condition (in other words, the prescription condition indicated by the prescription condition information acquired by the prescription condition information acquisition unit 22), the continuous packaging bag 3 having a strip-like shape including the continuously connected packaging bags 1 each of which contains the medicine is created. The continuous packaging bag 3 is introduced into the device main body 11 by the introduction part 13a formed in the housing 13 of the device main body 11 (S012).

The continuous packaging bag 3 introduced into the device main body 11 is moved by the conveyance unit 14 along the conveyance path 18 toward the downstream side in the conveyance direction. At this time, the continuous packaging bag 3 moves in a state where an end (tip) on the side having the empty bag 1A is located on the downstream side in the conveyance direction. When the continuous packaging bag 3 has moved to the downstream side in the conveyance direction, the empty bag 1A located on the tip side of the continuous packaging bag 3 passes between the light source 19a and the light receiver 19b of the measurement unit 19 in due course. At this time, the measurement unit 19 measures the light transmittance of the empty bag 1A as characteristic information of the packaging bag 1. Subsequently, the characteristic information indicating the light transmittance measured by the measurement unit 19 is sent to the processing device 12, and the characteristic information acquisition unit 28 of the processing device 12 acquires the characteristic information sent from the measurement unit 19.

Thereafter, the conveyance operation by the conveyance unit 14 is repeated intermittently (S014). With this configuration, after the empty bag 1A of the continuous packaging bag 3 has passed over the arrangement unit 15, the packaging bag 1 adjacent to the empty bag 1A will be disposed on the arrangement unit 15. Subsequently, each time the conveyance operation is performed, the packaging bag 1 in the continuous packaging bag 3, disposed on the arrangement unit 15 is switched. In each of conveyance operations, the continuous packaging bag 3 is conveyed by a predetermined amount to the downstream side in the conveyance direction.

During a period between one conveyance operation and the next conveyance operation (that is, during stoppage of the conveyance of the continuous packaging bag 3), the light irradiation unit 17 emits light to the medicine packed in the packaging bag 1 disposed on the arrangement unit 15 (S015). In this state, using each of cameras, namely, the first camera 16a and the second camera 16b, the image capturing unit 16 captures an image (that is, a verification target medicine) packed in the packaging bag 1 disposed on the arrangement unit 15 (S016).

The light irradiation step S015 by the light irradiation unit 17 will be described in detail. The light irradiation unit 17 emits light from one of the four light emitting units 17a, 17b, 17c, and 17d disposed around the arrangement unit 15, switches the light emitting units sequentially (S017 and S018), and then, emits light again from the newly switched light emitting unit 17a, 17b, 17c, or 17d. That is, the light irradiation unit 17 sequentially switches the light irradiation directions and emits light from each of directions. Subsequently, the image capturing unit 16 captures an image of the verification target medicine for each of light irradiation directions. This enables a total of eight images (the number of cameras x the number of light irradiation directions) to be captured for the medicine packed in the packaging bag 1 disposed on the arrangement unit 15.

The light irradiation step S015, the image capturing step S016, and the light emitting unit switching step S018 described above are repeatedly performed every time the packaging bag 1 disposed on the arrangement unit 15 is switched together with the conveyance operation.

The captured image is transmitted to the image acquisition unit 23 of the processing device 12 as needed (S019). Thereafter, preprocessing is performed by the preprocessing unit 24 of the processing device 12 on the image acquired by the image acquisition unit 23 (S020). This generates a preprocessed image that emphasizes edges of the identification information formed by engraving on the surface of the medicine.

Meanwhile, the verification unit 25 of the processing device 12 specifies a prescription condition for the medicine (that is, the verification target medicine) appearing in the preprocessed image (S021). Specifically, based on the prescription condition information acquired in S011, the verification unit 25 specifies the prescription conditions set for the medicine packaged in each of the packaging bags 1 in the continuous packaging bag 3 (specifically, the type and the number of medicine for each of types).

Thereafter, the verification unit 25 verifies the type and the number of the medicine packaged in each of the packaging bags 1 in the continuous packaging bag 3 following the procedure of the above-described verification flow (S022). In the verification flow, the verification unit 25 accesses the database DB of the server computer 70 and reads the master image corresponding to the prescription condition (specifically, the type of medicine) specified in the previous step S021. Subsequently, the verification unit 25 verifies the type and the number of medicines for each of types packed in the packaging bag 1 disposed on the arrangement unit 15, using the preprocessed image and the master image.

The above-described series of steps S014 to S022 from the conveyance operation to the verification flow is repeatedly executed every time the packaging bag 1 disposed on the arrangement unit 15 is switched until completion of the verification on the medicines in each of the packaging bags 1 in the continuous packaging bag 3 (S023).

The verification unit 25 performs the above-described verification with the medicine packaged in each of the packaging bags 1 in the continuous packaging bag 3 as a verification target. After completion of all verifications, the verification unit 25 displays character information indicating the result (verification result) on a display (S024). Specifically, the verification unit 25 displays, on the display, character information for notification of the verification result regarding the type of the medicine packed in each of the packaging bags 1 disposed on the arrangement unit 15.

The verification result to be displayed on the display may be displayed in any manner as long as it is possible to clearly grasp which packaging bag 1 in the continuous packaging bag 3 corresponds to the verification result for the medicine packed in the packaging bag 1. Accordingly, the verification results may be displayed by switching for each of the packaging bags, or verification result for each of the packaging bags 1 constituting the continuous packaging bag 3 may be displayed collectively in association with the position or order of the packaging bag 1.

On the other hand, the update processing unit 29 determines the necessity of executing the update processing following the above-described update flow (refer to FIG. 11) and executes the update processing when it is determined that the execution of the processing is necessary, so as to update the update target master image among the master images stored in the server computer 70 (S025).

Thereafter, the conveyance operation of the conveyance unit 14 allows the continuous packaging bag 3 to reach the discharge part of the housing 13 of the device main body 11, and the packaging bag 1 at the end of the continuous packaging bag 3 (the packaging bag 1 located most upstream in the conveyance direction) is discharged to the outside of the housing 13, and the basic operation of the medicine verification device 10 is completed at this point.

Note that determination of the necessity of executing the update processing according to the update flow, and the execution of the update processing when it is determined that the update processing is necessary may be performed after the continuous packaging bag 3 is discharged to the outside of the housing 13.

<<Effectiveness of the Medicine Verification Device According to the Present Embodiment>>

As described above, in determining the necessity of updating the master image, the medicine verification device 10 according to the present embodiment acquires characteristic information indicating the characteristic that influences the sharpness of the image (specifically, information illustrating the light transmittance) regarding the verified medicine packaging bag. Subsequently, the medicine verification device 10 executes the update processing for setting the image of the verified medicine as a master image only when the characteristic indicated by the acquired characteristic information (specifically, the light transmittance) satisfies a standard.

As described above, the medicine verification device 10 according to the present embodiment does not necessarily update the master image by setting the captured image of a medicine verified in the verification to match the type of medicine appearing in the master image as a new master image as performed in the verification method described in JP 2005-249615 A. Specifically, the necessity of updating the master image is determined in consideration of the sharpness of the captured image (that is, a candidate for a new master image). This makes it possible to perform update of the master image only in a case where the update is necessary (for example, where the update of the master image will be advantageous in subsequent determinations). As a result, the time required for updating can be reduced as compared with the case where the master image is constantly updated. Moreover, it is possible to save an image with higher sharpness as the master image, enabling subsequent determination of the type of medicines with high accuracy.

### Other Embodiments

While the medicine verification device and the medicine verification method of the present invention have been described above with reference to a specific example, the above embodiments are merely an example, and other embodiments are conceivable. For example, while the above embodiment is an exemplary case where a plurality of medicines is packaged in the packaging bag 1 and these medicines (a plurality of medicines) are medicines to be a verification target medicine, the present invention is not limited to this case. The number of medicines packaged in the packaging bag 1 can be set to any number and may be only one, or may be two or more.

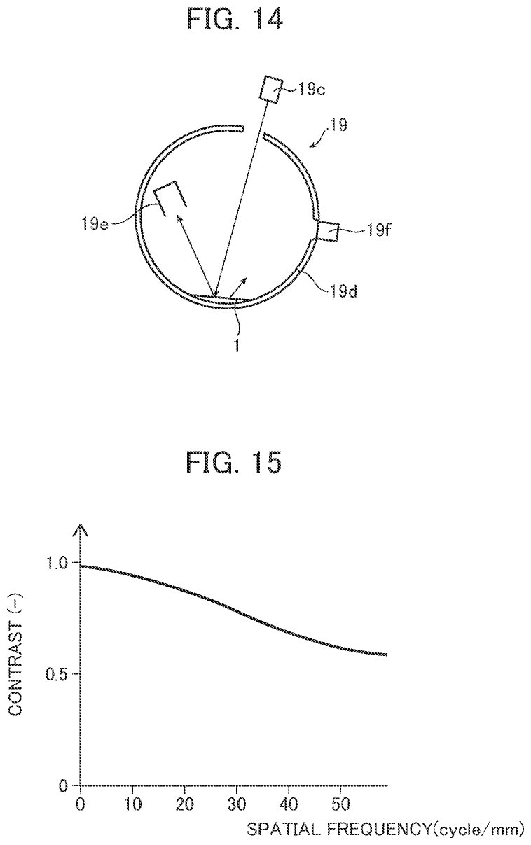

Furthermore, in the above embodiment, the optical characteristic (light transmissivity or light scattering characteristic) of the packaging bag 1 is measured by the measurement unit 19 provided inside the medicine verification device 10. More specifically, in the above embodiment, the measurement unit 19 measures the light transmittance of the packaging bag 1 inside the device main body 11 during conveyance of the continuous packaging bag 3. However, the present invention is not limited to this, and the optical characteristics of the packaging bag 1 may be measured outside the medicine verification device 10 using the measurement unit 19 illustrated in FIG. 14. FIG. 14 is a schematic diagram illustrating a method of measuring the optical characteristics of the packaging bag 1 using an integrating sphere.

The measurement unit 19 illustrated in FIG. 14 is a measuring device disposed outside the medicine verification device 10. The measurement unit 19 includes a light source 19c, a spherical integrating sphere 19d that is hollow and having an opening formed at a position facing the light source 19c, a light shield 19e disposed inside the integrating sphere 19d, and a detector 19f that detects the brightness inside the integrating sphere 19d. In the measurement unit 19 having such a configuration, the packaging bag 1 is disposed inside the integrating sphere 19d, and light is emitted from the light source 19c toward the packaging bag 1 through the opening of the integrating sphere 19d. The light applied onto the packaging bag 1 is emitted from the surface of the packaging bag 1 as scattered light and total reflection light. The total reflection light out of the emitted light is blocked by the light shield 19e. In contrast, the scattered light hits an inner wall surface of the integrating sphere 19d and repeats diffuse reflection. The brightness is eventually uniformized in the integrating sphere 19d so as to be detected by the detector 19f. Subsequently, based on the relationship between the brightness inside the integrating sphere 19d detected by the detector 19f and the amount of light emitted from the light source 19c, the light transmissivity or light scattering characteristics of the packaging bag 1, specifically, the light transmittance or a haze value is obtained.

In a case where the light transmissivity or light scattering characteristics of the packaging bag 1 has been measured outside the medicine verification device 10 by using the measurement unit 19 having the above configuration, the characteristic information indicating the measurement result is to be input from the measurement unit 19 to the processing device 12 of the medicine verification device 10, whereby the characteristic information acquisition unit 28 of the processing device 12 acquires the characteristic information indicating the optical characteristics of the packaging bag 1 measured outside the medicine verification device 10.

Furthermore, in the above embodiment, the characteristic information acquisition unit 28 of the processing device 12 acquires the characteristic information indicating light transmissivity such as light transmittance and the light scattering characteristic such as a haze value. However, the characteristic information is not limited to the above information. That is, the characteristic information may be any information as long as it is information indicating optical characteristics that influence the sharpness of the image captured by the image capturing unit 16. For example, the information may be a Modulation Transfer Function curve (hereinafter, the MTF curve) indicating the correspondence relationship between contrast and spatial frequency illustrated in FIG. 15. FIG. 15 is a diagram illustrating an example of the MTF curve. The MTF curve of the packaging bag 1 can be measured using a known measurement method (for example, the square wave chart method).

Note that the characteristic information acquisition unit 28 preferably acquires the characteristic information indicating at least one of the light transmittance, the haze value, and the MTF curve of the packaging bag 1. Furthermore, the characteristic information may include optical characteristics other than these.

Furthermore, the above-described embodiment has described an exemplary case of the medicine verification device 10 used to inspect the medicines packaged in the packaging bag 1 when the pharmacist prescribes the medicines to patients. However, the use of the medicine verification device 10 is not limited to the above-described use. The medicine verification device 10 may be utilized for the purpose of grasping the type and quantity (more precisely, the quantity of each of types) of the medicines by operators in a facility like a hospital when the medicines are brought by a patient in the packaging bag 1 when admitted to the facility.

What is claimed is:

1. A medicine verification device comprising:
an image capturing unit configured to capture an image of a verification target medicine packed in a packaging material having light transmissivity;
a verification unit configured to verify a type of the verification target medicine using a master image registered in association with the type of medicine and the image of the verification target medicine captured by the image capturing unit;
a characteristic information acquisition unit configured to acquire characteristic information indicating an optical characteristic that influences sharpness of the image captured by the image capturing unit, for the packaging material that packs the verification target medicine verified by the verification unit to have a type that matches the type of the medicine appearing in the master image; and
an update processing unit configured to execute update processing for updating the registered master image to the image of the verification target medicine,
wherein, in a case where the verification unit has verified that the type of the verification target medicine matches the type of the medicine appearing in the master image, the update processing unit determines whether the optical characteristic indicated by the characteristic information acquired by the characteristic information acquisition unit satisfies a standard for the optical characteristic, and executes the update processing when the optical characteristic satisfies the standard.

2. The medicine verification device according to claim 1,
wherein the characteristic information acquisition unit acquires the characteristic information indicating light transmittance of the packaging material, and
in a case where the verification unit has verified that the type of the verification target medicine matches the type of the medicine appearing in the master image, the update processing unit determines whether the light transmittance indicated by the characteristic information acquired by the characteristic information acquisition unit exceeds a threshold, and executes the update processing when the light transmittance exceeds the threshold.

3. The medicine verification device according to claim 1, further comprising
a measurement unit configured to measure the optical characteristic of the packaging material,
wherein the measurement unit is provided inside the medicine verification device, and
the characteristic information acquisition unit acquires the characteristic information indicating the optical characteristic measured by the measurement unit.

4. The medicine verification device according to claim 1, further comprising
a measurement unit configured to measure the optical characteristic of the packaging material, the measurement unit being provided outside the medicine verification device,
wherein the characteristic information acquisition unit acquires the characteristic information indicating the optical characteristic measured by the measurement unit by an input of the characteristic information indicating the optical characteristic measured by the measurement unit, into the medicine verification device.

5. The medicine verification device according to claim 3,
wherein the packaging material is a bag-shaped packaging bag,
the medicine verification device further comprises a conveyance unit configured to convey a strip-shaped continuous packaging bag including continuously arranged packaging bags along a conveyance path,
the image capturing unit captures an image for each of the packaging bags at an intermediate position of the conveyance path, and
the measurement unit measures the optical characteristic for at least one packaging bag of the continuous packaging bag at an intermediate position of the conveyance path.

6. The medicine verification device according to claim 5, further comprising a light irradiation unit configured to emit light toward the packaging bag within an imaging range of the image capturing unit, of the continuous packaging bag, when the image capturing unit captures the image.

7. The medicine verification device according to claim 5,
wherein the continuous packaging bag includes an empty packaging bag containing no medicine, and
the measurement unit measures the optical characteristic of the empty packaging bag.

8. The medicine verification device according to claim 2,
wherein, in a case where the verification unit has verified that the type of the verification target medicine matches the type of the medicine appearing in the master image, the update processing unit determines whether the light transmittance indicated by the characteristic information acquired by the characteristic information acquisition unit exceeds a threshold and the light transmittance of the packaging material appearing in the master image, and executes the update processing when the light transmittance indicated by the characteristic information acquired by the characteristic information acquisition unit exceeds the threshold and the light transmittance of the packaging material appearing in the master image.

9. The medicine verification device according to claim 2,
wherein, in a case where the verification unit has verified that the type of the verification target medicine matches the type of the medicine appearing in the master image, the update processing unit determines whether the light transmittance indicated by the characteristic information acquired by the characteristic information acquisition unit exceeds the threshold and whether an imaging position of the verification target medicine is closer to a center of an imaging range of the image capturing unit than an imaging position of the medicine appearing in the master image, and the update processing unit executes the update processing when the light transmittance indicated by the characteristic information acquired by the characteristic information acquisition unit exceeds the threshold and the imaging position of the verification target medicine is closer to the center of the imaging range of the image capturing unit than the imaging position of the medicine appearing in the master image.

10. The medicine verification device according to claim 1, wherein the characteristic information acquisition unit acquires the characteristic information indicating at least one of light transmissivity and a light scattering characteristic of the packaging material.

11. The medicine verification device according to claim 10, wherein the characteristic information acquisition unit acquires the characteristic information indicating at least one of light transmittance, a haze value, and a Modulation Transfer Function curve of the packaging material, the Modulation Transfer Function curve indicating a correspondence relationship between a contrast and a spatial frequency.

12. The medicine verification device according to claim 1, further comprising
a prescription condition acquisition unit configured to acquire prescription condition information indicating a prescription condition set for prescribing a medicine,
wherein the verification unit verifies the type of the verification target medicine using the master image corresponding to the type of the medicine specified by the prescription condition information and the image of the verification target medicine captured by the image capturing unit.

13. A medicine verification method comprising:
a step of capturing an image of a verification target medicine packed in a packaging material having light transmissivity by using an image capturing unit;
a step of verifying a type of the verification target medicine using a master image registered in association with the type of medicine and the image of the verification target medicine captured by the image capturing unit;
a step of acquiring characteristic information indicating an optical characteristic that influences sharpness of the image captured by the image capturing unit, for the packaging material that packs the verification target medicine verified to have a type that matches the type of the medicine appearing in the master image; and
a step of executing update processing for updating the registered master image to the image of the verification target medicine,
wherein, in a case where it is verified that the type of the verification target medicine matches the type of the medicine appearing in the master image, determination is made as to whether the optical characteristic indicated by the characteristic information acquired for the packaging material packing the verification target medicine satisfies a standard for the optical characteristic, and the update processing is executed when the optical characteristic satisfies the standard.

14. The medicine verification device according to claim 2, further comprising
a measurement unit configured to measure the optical characteristic of the packaging material,
wherein the measurement unit is provided inside the medicine verification device, and
the characteristic information acquisition unit acquires the characteristic information indicating the optical characteristic measured by the measurement unit.

15. The medicine verification device according to claim 2, further comprising
a measurement unit configured to measure the optical characteristic of the packaging material, the measurement unit being provided outside the medicine verification device,
wherein the characteristic information acquisition unit acquires the characteristic information indicating the optical characteristic measured by the measurement unit by an input of the characteristic information indicating the optical characteristic measured by the measurement unit, into the medicine verification device.

16. The medicine verification device according to claim 6,
wherein the continuous packaging bag includes an empty packaging bag containing no medicine, and
the measurement unit measures the optical characteristic of the empty packaging bag.
